(19) United States
(12) Patent Application Publication
(10) Pub. No.: US 2015/0329477 A1
(43) Pub. Date: Nov. 19, 2015

(54) CONTINUOUS METHOD FOR SEPARATING SALTS IN THE PRODUCTION OF DIMETHYLACETAMIDE

(71) Applicant: BASF SE, Ludwigshafen (DE)

(72) Inventors: Ortmund LANG, Quirnbach (DE); Norbert ASPRION, Ludwigshafen (DE); Axel BINDER, Karlsruhe (DE); Bernd METZEN, Westhofen (DE); Christof Wilhelm WIGBERS, Mannheim (DE); Petr KUBANEK, Mannheim (DE); Thomas STEINHILBER, Ludwigshafen (DE)

(73) Assignee: BASF SE, Ludwigshafen (DE)

(21) Appl. No.: 14/653,094

(22) PCT Filed: Dec. 18, 2013
(86) PCT No.: PCT/EP2013/077087

§ 371 Date: Jun. 17, 2015

(30) Foreign Application Priority Data
Dec. 19, 2012 (EP) 12197998.3

(51) Int. Cl. C07C 231/24; B01D 3/42; B01D 3/06

(52) U.S. Cl. CPC C07C 231/24; B01D 3/06; B01D 3/42

(57) ABSTRACT

What is proposed is a continuous process for discharging a solid, salt-containing phase comprising alkali metal acetates and/or alkaline earth metal acetates from the product mixture from the preparation of N,N-dimethylacetamide (DMAC) by reaction of methyl acetate (MeOAc) with dimethylamine (DMA) in the presence of a catalyst comprising N,N-dimethylacetamide (DMAC), methyl acetate (MeOAc), dimethylamine (DMA) and a catalyst, having the following process steps:
level-regulated feeding of the product mixture as feed stream into an evaporation vessel of a forced circulation evaporator,
flash evaporation of volatile components of the product mixture in the forced circulation evaporator to form a vapor phase comprising N,N-dimethylacetamide (DMAC) and precipitation of a solid, salt-containing phase comprising alkali metal acetates and/or alkaline earth metal acetates,
recycling of the volatile components of the product mixture obtained after the flash evaporation,
removal of a vapor phase comprising N,N-dimethylacetamide (DMAC) from the evaporation vessel as output stream,
concentration of the solid, salt-containing phase comprising alkali metal acetates and/or alkaline earth metal acetates in the forced circulation evaporation circuit of the forced circulation evaporator,
discharge of a substream comprising the solid, salt-containing phase comprising alkali metal acetates and/or alkaline earth metal acetates from the forced circulation evaporation circuit of the forced circulation evaporator,
solid/liquid separation of the discharged substream in at least one separation apparatus into a solid, salt-containing phase comprising alkali metal acetates and/or alkaline earth metal acetates and a liquid phase,
recycling of the liquid phase obtained after the solid/liquid separation into the forced circulation evaporation circuit as recycle stream,
wherein
the recycling of the volatile components of the product mixture obtained after the flash evaporation and of the solid, salt-containing phase comprising alkali metal acetates and/or alkaline earth metal acetates into the evaporation vessel is effected via an introduction section which ends within a range from 30 cm above the level surface to 20 cm below the level surface of the fill level of the evaporation vessel.

The invention relates to a continuous process for removing salts in the course of preparation of dimethylacetamide.

Dimethylacetamide (DMAC) finds use as a polar solvent, especially for polymers and gases, as a stripping agent, extractant and crystallization auxiliary. In the paints industry, DMAC, because of its high boiling temperature, is used for specific coating materials based on polymeric binders, especially polyamides and polyurethanes. DMAC additionally finds use for production of fibers and films and as a reaction medium. DMAC can be used as an auxiliary in the spinning of Spandex® fibers and can subsequently also be at least partly recovered.

WO 2006/061159 A1 discloses a continuous process for preparing N,N-dimethylacetamide (DMAC) by continuous reaction of methyl acetate (MeOAc) with dimethylamine (DMA) in the presence of a basic catalyst. The catalyst is in homogeneous and/or suspended form in the reaction mixture. When methanolic MeOAC solution is used, as obtained in the preparation of polytetrahydrofuran (poly-THF), it is also possible for by-products to be present. These by-products may especially be tetrahydrofuran (THF) and/or dimethyl ether. The liquid reaction outputs from the process can be decompressed in a distillation column for further workup.

Preferably, the basic catalyst present in the reaction output is neutralized. This is accomplished by addition especially of water or an aqueous or anhydrous protic acid, especially sulfuric acid, methanesulfonic acid, carboxylic acid, phosphoric acid and the like.

WO 2006/061159 A1 discloses a removal of salts from the reaction output by evaporation.

A disadvantage in this process is growing encrustation with increasing operating time, especially crystallization fouling and caking, especially on heated walls, which results in lower heat transfer performance, and also in blockage of pipelines and in that case ultimately a time-consuming and material-intensive exchange of system elements. The exchange of the system elements causes maintenance shutdowns with production shutdown periods, and also a high expenditure of material and the associated costs.

A further disadvantage is the cleaning and processing of the exchanged system elements. The time-consuming cleaning operation additionally gives rise to salt-containing wastewaters which may especially also contain residual amounts of DMAC, which, in addition to discontinuous occurrence of highly concentrated wastewater in a wastewater cleaning system, can lead to further problems.

It was therefore an object of the invention to provide an improved process which overcomes the above disadvantages.

The object is achieved by a continuous process for discharging a solid, salt-containing phase comprising alkali metal acetates and/or alkaline earth metal acetates from the product mixture from the preparation of N,N-dimethylacetamide (DMAC) by reaction of methyl acetate (MeOAc) with dimethylamine (DMA) in the presence of a catalyst comprising N,N-dimethylacetamide (DMAC), methyl acetate (MeOAc), dimethylamine (DMA) and a catalyst, having the following process steps:

level-regulated feeding of the product mixture as feed stream into an evaporation vessel of a forced circulation evaporator, where the forced circulation evaporator has, in flow direction, at least one evaporation vessel, a pump, a first heat exchanger and a recycle line into the evaporation vessel as a forced circulation evaporation circuit, where the recycle line has a throttle element and, disposed at the end in flow direction, an introduction section, where the level-regulated feeding of the product mixture is used for closed-loop control of a defined fill level in the evaporation vessel, where the product mixture at their defined fill level of the evaporation vessel has a level surface,

flash evaporation of volatile components of the product mixture in the forced circulation evaporator to form a vapor phase comprising N,N-dimethylacetamide (DMAC) and precipitation of a solid, salt-containing phase comprising alkali metal acetates and/or alkaline earth metal acetates,

recycling of the volatile components of the product mixture obtained in the vapor phase after the flash evaporation, of any unevaporated components of the product mixture in the liquid phase and of the solid, salt-containing phase comprising alkali metal acetates and/or alkaline earth metal acetates into the evaporation vessel via the recycle line,

removal of the vapor phase comprising N,N-dimethylacetamide (DMAC) from the evaporation vessel as output stream,

concentration of the solid, salt-containing phase comprising alkali metal acetates and/or alkaline earth metal acetates in the forced circulation evaporation circuit of the forced circulation evaporator,

discharge of a substream comprising the solid, salt-containing phase comprising alkali metal acetates and/or alkaline earth metal acetates from the forced circulation evaporation circuit of the forced circulation evaporator,

solid/liquid separation of the discharged substream comprising the solid, salt-containing phase comprising alkali metal acetates and/or alkaline earth metal acetates in at least one separation apparatus into a solid, salt-containing phase comprising alkali metal acetates and/or alkaline earth metal acetates and a liquid phase,

recycling of the liquid phase obtained after the solid/liquid separation into the forced circulation evaporation circuit as recycle stream,

wherein the recycling of the volatile components of the product mixture obtained after the flash evaporation and of the solid, salt-containing phase comprising alkali metal acetates and/or alkaline earth metal acetates into the evaporation vessel via the recycle line is effected via an introduction section which ends within a range from 30 cm above the level surface to 20 cm below the level surface of the fill level of the evaporation vessel.

Level-regulated feeding of the product mixture in the context of present invention is understood to mean feeding of the product mixture into the evaporation vessel which is regulated as a function of a defined level in the evaporation vessel. If the level in the evaporation vessel is below the defined level, product mixture in particular is fed in until the defined level is attained.

A feed stream in the context of the present invention is in principle understood to mean any desired feed stream which supplies the process with starting raw material(s) in particular.

A forced circulation evaporator in the context of the present invention is understood to mean a circulation evaporator which utilizes a pump in particular in order to force the product mixture comprising volatile components to flow through the circulation evaporator. The forced circulation evaporation circuit is formed in flow direction by the evaporation vessel, the pump, the first heat exchanger and the recycle line into the evaporation vessel.

A recycle line in the context of the present invention is understood to mean a line which, in the forced circulation evaporation circuit, in flow direction, leads back from the first heat exchanger into the evaporation vessel. The recycle line may especially be a pipe, a hose.

A throttle element in the context of the present invention is understood to mean any device which generates a pressure differential in the recycle line from the upstream side to the downstream side of the throttle element in flow direction, the pressure in the recycle line being higher upstream of the throttle element in flow direction than the pressure downstream of the throttle element in flow direction, such that decompression takes place downstream of the throttle element in flow direction.

An introduction section in the context of the present invention is understood to mean any device through which volatile components of the product mixture obtained after the flash evaporation, any unevaporated components of the product mixture in a liquid phase and a solid, salt-containing phase comprising alkali metal acetates and/or alkaline earth metal acetates is introduced into the evaporation vessel via the recycle line. The introduction section may especially be a pipe section, a nozzle, a hose.

Recycling of the volatile components of the product mixture obtained after the flash evaporation in the context of present invention is understood to mean the recycling of components of the product mixture from the forced circulation evaporation circuit into the evaporation vessel. The recycling may especially be effected downstream of the throttle element in the recycle line through the introduction section.

Recycling of the liquid phase obtained after the solid/liquid separation in the context of present invention is understood to mean the recycling of the liquid phase obtained after the solid/liquid separation into the evaporation vessel. Recycling of components into the evaporation vessel fundamentally also affects the fill level of the evaporation vessel.

A defined fill level in the context of the present invention is understood to mean a defined fill height in the evaporation vessel. The defined fill level is affected especially by the feeding of the product mixture, the removal of the vapor phase and the discharge of a substream. The definition of the fill level is determined by the distance between the level surface of the fill level and the introduction section.

Level surface in the context of the present invention is understood to mean the level surface at the fill height in the evaporation vessel. The level surface may especially be the outer surface of a liquid phase, or of a foam phase, at the fill height in the evaporation vessel.

Separation apparatus in the context of present invention is in principle understood to mean any apparatus for separation of liquid and solid phases. The separation can especially be effected by means of filtration, especially cake-forming filtration, cartridge filtration, membrane filtration, ultrafiltration, surface filtration, depth filtration, centrifugal processes, screening processes, sedimentation processes in the earth's gravitational field or a centrifugal field.

An advantageous processes a continuous process in which the product mixture in the recycle line, downstream of the throttle element in flow direction, has a flow rate within a range from 0.5 to 4 m/s. Preference is given to flow rates of 1-3 m/s, particular preference to those in the range of 1.5-2.5 m/s.

Preferably, the product mixture in the recycle line, downstream of the throttle element in the continuous process, has a temperature in the range from 50 to 300° C. Preference is given to temperatures in the range of 80-250° C., particular preference to those in the range of 100-250° C.

Preferably, in the continuous process, the product mixture leaving the introduction section has a flow having a Reynolds number greater than 10⁴.

Preferably, in the continuous process, the product mixture comprising N,N-dimethylacetamide (DMAC) in the recycle line, downstream of the throttle element in flow direction, has such a pressure/temperature relationship that N,N-dimethylacetamide (DMAC) is in the vapor phase.

Preferably, the removed vapor phase is distilled by cooling in at least one second heat exchanger to obtain N,N-dimethylacetamide (DMAC).

Preferably, the throttle element in the continuous process is a pressure-retaining unit, a valve, a regulating valve, a slide valve, a diaphragm, a ring diaphragm, a nozzle, a flap, a pipe constriction, a hole or else a combination thereof.

Preferably, the throttle element in the continuous process is set up such that a pressure differential in a region upstream of the throttle element to a region downstream of the throttle element in flow direction is preferably greater than 0.1 bar.

Preferably, the evaporation for removal of salt in the evaporation vessel has a pressure in the range of 0.01-5 bar, more preferably in the range of 0.1-2 bar.

Preferably, the vapor phase removed from the evaporation vessel in the continuous process has a proportion in the range from 30% to 99% by weight of N,N-dimethylacetamide (DMAC), based on the total weight of the feed stream.

Preference is given to processes in which 50%-99%, more preferably 90%-99%, based on the total weight of the feed stream, is evaporated. Nonvolatile compounds and salt remain in the residue.

Preferably, the process is conducted continuously and especially has a mean residence time of the product of value in the range of 1-60 minutes, more preferably in the range of 30-60 minutes.

Preferably, the flash evaporation in the continuous process comprises a plurality of evaporation apparatuses arranged in series, in parallel, or in combinations thereof.

In a suitable configuration, the evaporation comprises a plurality of forced circulation evaporators arranged in series, in parallel or in combination thereof. The connection system of the forced circulation evaporators may comprise, for example, two to twelve, preferably two to ten and especially two, three, four, five or six, identical or different forced circulation evaporators. The forced circulation evaporators may each be operated with or without recycling. The output from one forced circulation evaporator may also be conducted at least partly into an upstream forced circulation evaporator.

Preferably, the flash evaporation takes place in one stage or a plurality of successive stages, for example in two, three, four, five or six successive stages.

The solid/liquid separation in the continuous process is a filtration, especially a cake-forming filtration, a cartridge filtration, a membrane filtration, an ultrafiltration, a surface filtration, a depth filtration, a centrifugal process, a screening process, a sedimentation process in the earth's gravitational field and/or a centrifugal field, or else combinations thereof.

The solid/liquid separation in the continuous process is continuous or batchwise.

The catalyst in the continuous process is a basic catalyst, alkali metal hydroxide, alkaline earth metal hydroxide, alkali metal alkoxide, alkaline earth metal alkoxide, alkali metal carbonate, alkaline earth metal carbonate, alkali metal hydrogencarbonate, alkaline earth metal hydrogencarbonate, an amine, especially a tertiary amine, and combinations thereof, and the alkali metal is Li, Na, K, Rb, Cs and combinations thereof.

The evaporation of the continuous process has a specific heating surface load in the range of 1-100 kW/m², based on the heating output transferred and the evaporator surface area of the evaporator.

Preference is given to a heating surface load in the range of 10-80 kW/m², most preferably in the range of 20-40 kW/m².

Preferably, the heat exchanger is heated by means of heating steam or heat carrier medium.

The process according to the invention has the following advantages:
No deposits or reduced deposits on the walls of the evaporation vessel.
In conventional processes, the evaporation vessel is heated, for example, from the outside and there is heating of the walls of the evaporation vessel and direct transfer of the heat energy from the walls to the product mixture in the evaporation vessel. The direct heat transfer from the heated walls of the evaporation vessel to the product mixture is accompanied by formation of deposits as a result of phase transitions on the wall surfaces that are in contact with the product mixture. In contrast, in accordance with the invention, heating of the product mixture takes place in the first heat exchanger and thus outside the evaporation vessel. The subsequent flash evaporation of the heated product mixture then takes place, in accordance with the invention, downstream of the throttle element in flow direction, i.e. only in the course of recycling of the heated product mixture into the evaporation vessel. Thus, the continuous process of the invention does not have any direct transfer of heat energy from the heated walls of the evaporation vessel to the product mixture, and no deposits or only slight deposits form on the wall surfaces of the evaporation vessel that are in contact with the product mixture.
No formation or reduced formation of a foam layer and/or crusts at the level surface in the evaporation vessel.
In conventional processes, a foam layer may form at first at the level surface, caused by the phase transition of the liquid phase of the product mixture to the adjoining atmosphere in the evaporation vessel. For this not to happen, the recycling of the components of the product mixture obtained after the flash evaporation into the evaporation vessel, according to the invention, is executed in such a way that it is effected at least partly in the region of the level surface. For this purpose, for example, a pipeline disposed partly above the level surface and partly directly at or below the level surface may be provided. In this way, the face transition between the liquid phase of the product mixture and the adjoining atmosphere in the evaporation vessel is disrupted continuously and at least a portion of the level surface is, for example, in motion, such that foam formation is disrupted and/or prevented. If foam formation, as a precursor of crust formation, encrustation and/or cake formation, is prevented, no crusts can form any longer at the level surface either. Thus, the process according to the invention can be operated continuously and no production shutdowns because of crust formation at the level surface are required.
In the course of cleaning of the evaporation vessel, no highly concentrated, salt-containing wastewater is obtained.
In conventional processes, deposits, caked material and crusts in an evaporation vessel are dissolved with a solvent, for example water. This gives rise to highly concentrated, salt-containing water in the cleaning of the evaporation vessels. In contrast, in the process according to the invention, at least a portion of the product mixture having a high salt content is discharged from the evaporation vessel and the solid is separated from the liquid with a separation apparatus. The liquid removed is fed back to the evaporation vessel as a second return stream and the solids can be discharged from the process, for example as waste. It is thus unnecessary to dispose of highly concentrated, salt-containing wastewater in the process according to the invention.
The recycling of the liquid phase obtained after the solid/liquid separation into the evaporation vessel achieves recycling of product of value and feeding thereof to the feed stream of the evaporation vessel. Thus, in contrast to conventional processes, the feed stream is enriched with the liquid phase obtained after the solid/liquid separation. In this way, savings in the feed stream can be implemented.
In conventional processes, the cleaning of the evaporation vessel is unavoidable since, as already detailed above, as a result of the process, deposits, caked material and encrustations form on the walls of the evaporation vessel. In the process according to the invention, in contrast, it is possible to actively influence the deposition tendency as described above. Firstly, the heating of the product mixture takes place outside the evaporation vessel in a first heat exchanger, as a result of which there is no direct heat transfer, for example, from heated walls of the evaporation vessel to the product mixture, as a result of which no deposits and/or only slight deposits as a result of phase transitions can form on the wall surfaces that are in contact with the product mixture. Secondly, the amount of depositable salts in the process according to the invention is actively influenced by discharge of substreams from the product mixture in the evaporation vessel and removal of the solids in a separation apparatus. Specifically the possibility according to the invention of combining these two influencing methods is particularly advantageous for a continuous, disruption-free and economically viable process regime with high throughput rates.

The invention is elucidated in detail hereinafter by a working example and a drawing.

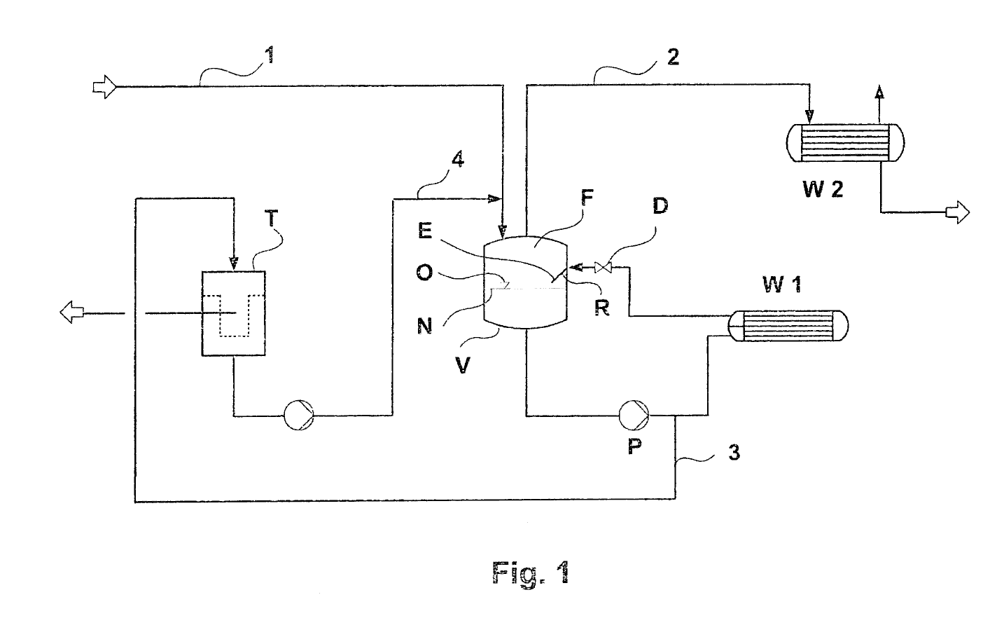

FIG. 1 shows a schematic overview diagram of the process according to the invention.

The following reference symbols are used:
D throttle element
E introduction section
F vapor phase
N fill level
O level surface
P pump
R recycle line
T separation apparatus
V evaporation vessel
W1 first heat exchanger
W2 second heat exchanger
1 feed stream
2 removal stream
3 substream
4 recycle stream

The schematic overview diagram in FIG. 1 with a forced circulation evaporator, a downstream distillation column and a solid/liquid separation apparatus T shows a preferred embodiment of the process according to the invention. The continuous process is fed via a feed stream 1. The level-regulated feeding of a product mixture as feed stream 1 is accompanied by the formation of a fill level N with a level surface O in an evaporation vessel V of the forced circulation evaporator. The forced circulation evaporator has, downstream of the evaporation vessel V in flow direction, a pump P, a first heat exchanger W1 and a recycle line R into the evaporation vessel V as a forced circulation evaporation circuit. From the evaporation vessel V, at the base, a stream of the product mixture is removed and fed to the first heat exchanger W1 with a pump P. The product mixture is heated in the first heat exchanger W1 and recycled via the recycle line R and an introduction section E disposed at the end in flow direction into the evaporation vessel V. The pumping of the product mixture in flow direction through the first heat exchanger W1 against the throttle element D builds up a pressure in the first heat exchanger which can be regulated particularly by means of the throttle element D. In the forced circulation evaporation circuit, downstream of the throttle element D in flow direction, there is flash evaporation of volatile components of the product mixture to form a vapor phase F comprising N,N-dimethylacetamide (DMAC) and precipitation of a solid, salt-containing phase. The volatile components of the product mixture obtained after the flash evaporation, any unevaporated components of the product mixture in a liquid phase and a solid, salt-containing phase are recycled into the evaporation vessel through the recycle line R via the introduction section E. The vapor phase F is removed from the evaporation vessel V as output stream 2 and condensed in a second heat exchanger W2. The evaporation operation and the removal of the vapor phase result in concentration of the solid, salt-containing phase in the evaporation vessel V. In the case of a concentrated salt content of the product mixture in the evaporation vessel V, a substream 3 is discharged from the evaporation vessel V and fed to the solid/liquid separation apparatus T, for example a suction filter. After the solids have been separated from the liquid in the solid/liquid separation apparatus T, liquid phase obtained is fed back to the evaporation, especially to the evaporation vessel V.

A preferred plant for performing the process according to the invention may have a plurality of forced circulation flash evaporators arranged in a cascade connection. In a cascade connection, forced circulation flash evaporators are arranged such that a residue from a first forced circulation flash evaporator is passed into a second forced circulation flash evaporator and the residue from the second forced circulation flash evaporator is passed into a third forced circulation flash evaporator, which is continued further with a further number of forced circulation flash evaporators. In the case of a plurality of evaporator stages, the vapors from the upstream evaporator stage are used to heat the downstream evaporator stage. Preferably, the vapors can also be sent to a distillation column.

Preferably, there is no formation of vapor bubbles when the product mixture is heated in the first heat exchanger W1. In this way, it is possible to avoid precipitation and the caking of solids that have a tendency to form crusts on heated walls. Vapor bubble formation does not take place until downstream of the throttle element D, especially a pressure-retaining valve, in flow direction.

Preferably, a separation is effected in the separation apparatus T by the customary processes, especially with cartridge filters. In this case, it is possible with preference to use an open filter fabric made from Teflon having an air passage rate of 150 L/dm²/min.

If the filtercake resistance becomes too high, the feed is stopped and the filtercake is cleaned further to remove organic residues by blowing with nitrogen or steam before the filtercake is either disposed of or the salt is dissolved with water and sent to a water treatment plant.

An advantage of using steam for the blowing-dry operation is the option of condensing the steam obtained with the organic material. The water obtained can then be used for catalyst breakdown and enables recycling of the organic material. In the case of use of nitrogen, the stream would have to be disposed of by means of a flare.

The output which has been evaporated off the solids and partially or totally condensed is worked up by distillation under the customary distillation conditions, for example in one or more columns connected to one another.

### WORKING EXAMPLE

The pilot plant comprised a forced circulation evaporator, a downstream distillation column and a solid/liquid separation apparatus. In the circuit of the forced circulation evaporator, in flow direction, an evaporation vessel V, a centrifugal pump P, a first heat exchanger W1 and a recycle line R the evaporation vessel V. Disposed downstream of the first heat exchanger W1 in the recycle line R was a throttle element D and, at the end in flow direction, an introduction section E. As feed stream 1, a product mixture was fed under level control into the evaporation vessel V. The product mixture was produced according to WO 2006/061159 A1, consisting of 15% by weight of methanol, 3% by weight of dimethylamine, 6% by weight of methyl acetate, 75% by weight of DMAC and about 1% by weight of sodium methoxide. 1% by weight of water was added continuously to the product mixture, based on the feed stream 1, in order to neutralize sodium methoxide, and the product mixture was conveyed under level control into the evaporation vessel V, in order to form a fill level N in the evaporation vessel having a level surface O. From the evaporation vessel V, the product mixture was pumped into the first heat exchanger W1 with a centrifugal pump P and heated to a temperature in the range from 120° C. to 150° C. The first heat exchanger W1 was heated by means of a heat carrier oil. The specific heating surface output was 20 kW/m².

The pumping of the product mixture through the first heat exchanger W1 in flow direction against a throttle element D built up a pressure in the first heat exchanger W1. This pressure in the heat exchanger was regulated with the throttle element, especially a regulating valve in the outlet region of the heat exchanger W1, and adjusted such that there was no vapor bubble formation and no evaporation in the heat exchanger W1 during the heating of the product mixture. The throttle element D was adjusted such that there is a pressure differential between the pressure within the heat exchanger and the pressure downstream of the throttle element D in flow direction within a range from 0.1 to 0.5 bar, with a drop in the pressure downstream of the throttle element in flow direction. The pressure in the evaporation vessel was in the range from 0.5 to 1.2 bar.

Downstream of the throttle element in flow direction, the volatile components of the product mixture were flash-evaporated to form a vapor phase F comprising N,N-dimethylacetamide (DMAC) and precipitate a solid, salt-containing phase comprising especially sodium acetate. The vapor phase F especially also comprises the components of the product mixture and may comprise, for example, about 15% by weight of methanol, about 3% by weight of dimethylamine, about 6% by weight of methyl acetate, about 75% by weight of DMAC, about 1% by weight of water, based on the total weight of the product mixture. The volatile components of the product mixture obtained after the flash evaporation, any unevaporated components of the product mixture in a liquid phase and a solid, salt-containing phase were recycled into the evaporation vessel through the recycle line R via the introduction section E. The flow rate in the recycle line R downstream of the throttle element D in flow direction was 2 m/s. The introduction section was adjusted such that it ends within a region from 30 cm immediately above to 20 cm below the level surface O of the fill level N of the evaporation vessel. The vapor phase was removed from the evaporation vessel as output stream 2 and condensed in the second heat exchanger W2. The evaporating operation and the removal of the vapor phase resulted in concentration of the solid, salt-containing phase in the evaporation vessel V. Concentrations up to a range of more than 80% by weight, preferably of more than 50% by weight, based on the total weight of the product mixture are possible. Over and above a salt concentration of 25% by weight, based on the total weight of the product mixture in the evaporation vessel V, a substream 3 was discharged from the evaporation vessel V under level control and fed to a solid/liquid separation apparatus T configured as a suction filter. After the solids had been separated from the liquid, the liquid was fed back to the evaporation, especially the evaporation vessel V. During the continuous three-week experiment, no solid deposits were found in the pipelines of the first heat exchanger W1. No cleaning of the pilot plant during the test period was necessary.

1. A continuous process for discharging a solid, salt-containing phase comprising alkali metal acetates and/or alkaline earth metal acetates from a product mixture from preparation of N,N-dimethylacetamide by reaction of methyl acetate with dimethylamine in the presence of a mixture comprising N,N-dimethylacetamide, methyl acetate, dimethylamine and a catalyst, the process comprising:
level-regulated feeding of the product mixture as feed stream (1) into an evaporation vessel of a forced circulation evaporator,
wherein
the forced circulation evaporator has, in a flow direction, at least one evaporation vessel, a pump, a first heat exchanger and a recycle line into the evaporation vessel as a forced circulation evaporation circuit,
the recycle line has a throttle element and, disposed at an end in flow direction, an introduction section,
the level-regulated feeding of the product mixture is used for closed-loop control of a defined fill level in the evaporation vessel, and
the product mixture at the defined fill level of the evaporation vessel has a level surface;
flash evaporation of volatile components of the product mixture in the forced circulation evaporator to form a vapor phase comprising N,N-dimethylacetamide and precipitation of a solid, salt-containing phase comprising one or more alkali metal acetate, alkaline earth metal acetate, or both;
recycling of the volatile components of the product mixture obtained in the vapor phase after the flash evaporation, of any unevaporated components of the product mixture in a liquid phase and of the solid, salt-containing phase comprising the one or more alkali metal acetate, alkaline earth metal acetate, or both into the evaporation vessel via the recycle line;
removal of the vapor phase comprising N,N-dimethylacetamide from the evaporation vessel as output stream (2);
concentration of the solid, salt-containing phase comprising the one or more alkali metal acetate, alkaline earth metal acetate, or both in the forced circulation evaporation circuit of the forced circulation evaporator;
discharge of a substream (3) comprising the solid, salt-containing phase comprising the one or more alkali metal acetate, alkaline earth metal acetate, or both from the forced circulation evaporation circuit of the forced circulation evaporator;
solid/liquid separation of the discharged substream (3) comprising the solid, salt-containing phase comprising the one or more alkali metal acetate, alkaline earth metal acetate, or both in at least one separation apparatus into a solid, salt-containing phase comprising the one or more alkali metal acetate, alkaline earth metal acetate, or both; and
recycling of the liquid phase obtained after the solid/liquid separation into the forced circulation evaporation circuit as recycle stream (4),
wherein the recycling of the volatile components of the product mixture obtained after the flash evaporation, of any unevaporated components of the product mixture in a liquid phase and of the solid, salt-containing phase comprising the one or more alkali metal acetate, alkaline earth metal acetate, or both into the evaporation vessel via the recycle line is effected via an introduction section which ends within a range from 30 cm above the level surface to 20 cm below the level surface of the fill level of the evaporation vessel.

2. The process of claim 1, wherein the product mixture in the recycle line downstream of the throttle element in the flow direction, has a flow rate within a range from 0.5 to 4 m/s.

3. The process of claim 1, wherein the product mixture in the recycle line downstream of the throttle element has a temperature in the range from 50 to 300° C.

4. The process of claim 1, wherein the product mixture leaving the introduction section has a flow having a Reynolds number greater than 10⁴.

5. The process of claim 1, wherein the product mixture comprising the N,N-dimethylacetamide in the recycle line, downstream of the throttle element in the flow direction, has such a pressure/temperature relationship that the N,N-dimethylacetamide is in the vapor phase.

6. The process of claim 1, wherein the removed vapor phase is distilled by cooling in at least one second heat exchanger to obtain N,N-dimethylacetamide.

7. The process of claim 1, wherein the throttle element is a valve, a slide valve, a diaphragm, a ring diaphragm, a nozzle, a flap, a pipe constriction, a hole or a combination thereof.

8. The process of claim 1, wherein the throttle element is set up such that a pressure differential in a region upstream of the throttle element to a region downstream of the throttle element in the flow direction is greater than 0.1 bar.

9. The process of claim 1, wherein the removed vapor phase from the evaporation vessel has a proportion in the range from 30% to 99% by weight of N,N-dimethylacetamide, based on a total weight of the feed stream (1).

10. The process of claim 1, wherein the flash evaporation comprises a plurality of evaporation apparatuses arranged in series connection, in parallel connection or in combinations thereof.

11. The process of claim 1, wherein the solid/liquid separation comprises a filtration.

12. The process of claim 1, wherein the solid/liquid separation is continuous or batchwise.

13. The process of continuous process according to claim 1, wherein the first heat exchanger (W1) has a specific heating surface load in the range from 1 to 100 kW/m².
